(19) United States
(12) Patent Application Publication
(10) Pub. No.: US 2025/0136968 A1
(43) Pub. Date: May 1, 2025

(54) METHODS FOR SEPARATION AND/OR PURIFICATION OF NUCLEIC ACIDS

(71) Applicant: QUANTOOM BIOSCIENCES S.A., Nivelles (BE)

(72) Inventors: Sébastien DALMAT, Nivelles (BE); Emilie BOULANGER, Nivelles (BE); José CASTILLO, Nivelles (BE)

(21) Appl. No.: 18/912,444

(22) Filed: Oct. 10, 2024

Related U.S. Application Data
(63) Continuation of application No. PCT/EP2023/059818, filed on Apr. 14, 2023.

(30) Foreign Application Priority Data
Apr. 15, 2022 (EP) EP22168645.4
Jan. 20, 2023 (EP) EP23152754.0

(51) Int. Cl. C12N 15/10; C07H 1/08

(52) U.S. Cl. CPC C12N 15/1013; C07H 1/08

(57) ABSTRACT

The disclosure relates to methods for nucleic acid purification, comprising (a) combining a sample comprising at least one nucleic acid with a binding buffer comprising at least one silica-based magnetic particle and having a pH ranging from 5 to about 10 to form a solution; (b) incubating the sample with the binding buffer for a time period sufficient to reversibly bind the at least one nucleic acid to the magnetic particle(s) to form at least one modified magnetic particle, (c) separating the at least one modified magnetic particle from the solution, (d) washing the at least one modified magnetic particle with at least one wash buffer; and (e) combining the at least one modified magnetic particle with a low conductivity elution buffer to elute the purified nucleic acid from the magnetic particle. Kits comprising these buffers and magnetic particles are also disclosed herein.

### CROSS-REFERENCE

This application is a Continuation of International Application No. PCT/EP2023/059818, filed Apr. 14, 2023, which claims the benefit of EP Application Serial No. 22168645.4, filed Apr. 15, 2022, and EP Application Serial No. 23152754.0, filed Jan. 20, 2023, each of which is incorporated herein by reference in its entirety.

### FIELD OF THE INVENTION

The present invention relates to general methods and kits for nucleic acid purification and, more particularly, to silico-based magnetic particle-based kits and methods for purifying samples containing RNA.

### BACKGROUND

Nucleic acid purification and isolation is an important step in various biochemical and diagnostic procedures. In vitro transcription (IVT) can provide a way to in vitro transcribe nucleotides with desired sequences or modifications. The IVT-obtained mRNA can be further used for RNA based therapeutics such as vaccine formulation. However, it may be necessary to purify post-IVT samples to remove one or more contaminants, such as salts, proteins, enzymes, oligonucleotides, and the like, before proceeding with downstream applications. The presence of such contaminating materials can impede or prevent many downstream processes. Thus, it can be important to effectively isolate nucleic acids from the post-IVT mixture to ensure a desired end-use functionality.

Nucleic acid purification methods are described in the art. Traditional precipitation procedures such as phenol/chloroform extraction yield high purity RNA but are time-consuming and complex. Solid phase-based methods, such as methods utilizing magnetic beads, spin columns, and/or filtration systems, have been presented as an alternative solution. However, methods discussed above present some disadvantages, such as slow speed, high complexity, and poor overall yield.

Nucleic acid purification methods that are safe for use as medicament is even more limited. Current RNA vaccine production protocols use expensive technologies such as chromatography or tangential flow filtration for purification steps. Magnetic beads-based RNA purification processes are also used in the area of RNA production but mainly for analytical purposes due to the necessity of the use of toxic and still expensive substances in the process, such as chaotropic agent guanidinium thiocyanate and solvents like isopropanol, which is limiting their accessibility and utilization on RNA-base therapies.

To offer a larger access to RNA-based medicine, there is a need for purification methods that are efficient, cheaper and compatible with drug-safety regulations. Current disclosure aims to provide a solution to this.

### SUMMARY OF THE INVENTION

The present invention and embodiments thereof serve to provide a solution to one or more of above-mentioned disadvantages and describes novel bead-based purification methods for nucleic acid sequences using common, non-dangerous and affordable substances/technologies.

The disclosure relates, in various embodiments, to methods for purifying nucleic acids. In an embodiment, said method is a method as disclosed in claim 1.

Said method comprises (a) combining a sample comprising at least one nucleic acid of interest in a binding buffer having a pH ranging from about 5 to about with silica-based magnetic particles to form a solution, (b) incubating the solution for a time period sufficient to reversibly bind the at least one nucleic acid to the magnetic particles to form modified magnetic particles, (c) separating the modified magnetic particles from the combined solution by applying a magnetic field, (d) washing the at least one modified magnetic particle with at least one wash buffer; and (e) combining the modified magnetic particle with an elution buffer in order to allow the elution of said nucleic acid of interest from said magnetic particle, wherein said elution buffer has a pH ranging from about 5 to 10 and wherein said elution buffer has a conductivity of between 0.001 and 40 mS/cm or wherein the total salt concentration is from 0 to 50 mM.

Further embodiments are described in the dependent claims.

According to various embodiments, the binding buffer can comprise at least one salt, preferably a safe for human usage, more preferably a common salt, present in a concentration range from around 0.1 M to around 5 M and at least one alcohol in a concentration range from around 10% to 50% volume per volume (v/v) and/or polyethylene glycol (PEG) in a concentration range of 10% to 40% (v/v).

In other embodiments, the wash buffer can comprise at least one alcohol in about 60 to 80% v/v and optionally at least one buffer compound, such as Tris-HCl or Sodium citrate.

Said elution buffer is preferably chosen from water and solutions comprising at least one buffer compound, such as Tris-HCl or Sodium citrate and/or at least one ion chelating agent, e.g., EDTA and alike. Silica-based magnetic particles, can be coated with one or more ligand interacting with said nucleic acid, and combinations thereof.

Also disclosed herein are nucleic acid purification kits comprising these buffers and magnetic particles as disclosed in claim 19.

### DESCRIPTION OF FIGURES

FIG. 1 shows a work-flow of nucleic acid purification method according to embodiments of the present invention.

FIG. 2 and FIG. 3 show two example processes of a nucleic acid purification method according to embodiments of the current invention.

FIG. 4A and FIG. 4B and FIG. 5A and FIG. 5B show a set of graphs showing successful high quality RNA purification according to the method disclosed, using binding buffer in different NaCl concentrations and pH.

### DETAILED DESCRIPTION OF THE INVENTION

### Definitions

Unless otherwise defined, all terms used in this disclosure, including technical and scientific terms, have the meaning as commonly understood by one of ordinary skill in the art to which this disclosure belongs. By means of further guidance, term definitions are included to better appreciate the teaching of the present disclosure.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosure. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g., component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

As used herein the term “magnetic particle” and variations thereof is intended to denote a particle with a magnetic, e.g., paramagnetic or superparamagnetic, core coated with at least one material having a surface to which nucleic acid can reversibly bind.

The terms “magnetic particle” and “magnetic bead” are used interchangeably.

The term “biological sample” refers to a sample derived from a biological origin, such as, bacteria and other microorganisms, bacteria, from plants, humans or non-human animals, preferably warm-blooded animals, even more preferably mammals, such as, e.g., non-human primates, rodents, canines, felines, equines, ovines, porcines, and the like. The term “non-human animals” includes all vertebrates, e.g., mammals, such as non-human primates, (particularly higher primates), sheep, dog, rodent (e.g., mouse or rat), guinea pig, goat, pig, cat, rabbits, cows, and non-mammals such as chickens, amphibians, reptiles etc. Preferably, the biological sample is derived from human origin. The biological sample can be a biological fluid or a non-fluid biological sample.

Unless otherwise defined, all terms used in this disclosure, including technical and scientific terms, have the meaning as commonly understood by one of ordinary skill in the art to which this disclosure belongs. By means of further guidance, definitions for the terms used in the description are included to better appreciate the teaching of the present disclosure. The terms or definitions used herein are provided solely to aid in the understanding of the disclosure.

Reference throughout this specification to “one embodiment” or “an embodiment” means that a particular feature, structure, or characteristic described in connection with the embodiment is included in at least one embodiment of the present disclosure. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure, and form different embodiments, as would be understood by those in the art. For example, in the following claims, any of the claimed embodiments can be used in any combination.

### DETAILED DESCRIPTION

The current invention aims to provide methods and kits that allow the purification of nucleic acids by means of magnetic particles, wherein once eluted, the nucleic acids are already in a buffer suitable for further processing and formulating, without the need for any further buffer exchange. The methods and kits allow a (cost) efficient purification of said nucleic acids. Here disclosed bead-based purification methods allow to produce RNA-based medicine complying with the authority's requirement by employing common, non-dangerous and affordable substances/technologies. To perform these processes, preferably common salts such as sodium chloride, buffers such as Tris-HCl or citrate and limited quantities of solvent are used. The method described here allows to purify acid nucleic with great yield (within 80-100%) and sufficient purity. The RNA integrity is conserved while reducing the use of hazardous agent(s) and making the technology affordable.

In a first aspect, the current invention, relates to a method for nucleic acid purification. In an embodiment said method comprises:
a. combining a sample comprising at least one nucleic acid of interest in a binding buffer having a pH ranging from 5 to 10 with silica-based magnetic particles to form a solution;
b. incubating the solution for a time period sufficient to reversibly bind the at least one nucleic acid to the magnetic particles to form modified magnetic particles;
c. separating the modified magnetic particles from the combined solution by applying a magnetic field;
wherein the binding buffer comprises at least one salt, present in a concentration ranging from 0.1 M to 5 M, at least one first alcohol, present in a concentration between 10% to 50% v/v and/or PEG at a concentration of 10% to 40% v/v,
d. washing the at least one modified magnetic particle with at least one wash buffer comprising at least one second alcohol at a concentration of between 60% to 100%; and
e. combining the modified magnetic particle with an elution buffer in order to allow the elution of said nucleic acid of interest from said magnetic particle, wherein said elution buffer has a pH ranging from about 5 to 10 and wherein said elution buffer has a conductivity of from 0.001 and 40 mS/cm or wherein the total salt concentration is from 0 to 50 mM.

It is understood that the separation of magnetic particles from the solution also entails physical separation, as in removing the supernatant from the modified magnetic particles.

In embodiments, the washing of the modified particles can be repeated several times, for example, 2 times, 4 times, 5 times, 6 times 7 times, 8 times, 9 times, and 10 times.

It will be clear to a skilled person that washing entails both addition and removal of the wash buffer to the modified beads. In each successive washing step, modified particles are separated from the wash buffer by means of magnetic separation before proceeding with the next washing and/or before moving to any other following step such as the elution step.

It should be also clear to the skilled person that in embodiments, the elution step is repeated multiple times to increase the yield. For example, the elution is repeated 2 times, 4 times, 5 times, 6 times 7 times, 8 times, 9 times, and 10 times.

Although the step-wise purification protocol is similar to prior art, the method present here uses novel combination of the buffers where the use of toxic or unwanted compounds commonly used in the prior art (e.g., guanidine salts) are eliminated, the production yield is increased, the cost is decreased, and the time to complete the RNA purification is shortened. The effect is mainly achieved by the choice of the specific type of magnetic particles, being silica-based magnetic particles. Said silica-based magnetic particles allows the use of typically less harsh binding and especially elution conditions, while still ensuring a high yield and high purity of the nucleic acids to be purified. The resulting purified sample can immediately be used downstream for further pharmaceutical formulation or use in the production of a pharmaceutical formulation without the need for further downstream purification to remove components used in the binding and/or elution buffer. This is a major difference compared to known prior art methods, that e.g. use cellulose or cellulose derivatives.

The method is particularly advantageous in efficient purification of nucleic acid from unwanted contaminants of said sample (e.g., salts, proteins, enzymes, oligonucleotides, carbohydrates, DNA templates, and nucleotide triphosphates (NTP)). This method allows to purify said nucleic acid, wherein said nucleic acid is RNA, more specifically in vitro transcribed RNA, with high yield (within 80-100%) and sufficient purity and the purified RNA integrity is conserved while reducing the use of hazardous and expensive agent(s) such as chaotropic agent(s) like guanidinium thiocyanate which is commonly used in practice. Overall, making the technology safe and affordable.

In specific embodiments, the method can comprise adding a further sample comprising the at least one nucleic acid of interest in the binding buffer and optionally non-modified silica-based magnetic particles to the modified magnetic particles of a preceding step c to form a solution, and repeating steps b and c. As such, the method disclosed here comprises repetition of some consecutive steps.

In embodiments, the steps comprising sample-magnetic particle binding (step a and b) and consecutive magnetic separation (step c), hereafter referred as “binding steps”, of the method can be successively repeated several times before proceeding with the next steps comprising washing and elution. As such, in embodiments, steps a to c of the present method can be repeated one or more times before proceeding to step d, preferably 1 to 20 times, more preferably 1 to 10 times. In embodiments, the biding steps are successively repeated up to 20 times, up to times, up to 10 times, up to 9 times, up to 8 times, up to 7 times, up to 6 times, up to 5 times, up to 4 times, up to 3 times, up to 2 times, preferably between 5 to times. Inventors found that the repetition led to reduction in use of alcohol such as ethanol per unit of nucleic acids purified.

In embodiments, cumulative repeated binding steps can reduce the total alcohol consumption by 95%, 90%, 85%, 80%, 75%, 70%, 60%, 50%, 40%, 30%, 20%, 10%, 5% per unit of nucleic acid compared to the method where single binding step is applied. In embodiments the alcohol can be ethanol.

According to embodiments, the reduction in use of alcohol when successive binding steps are applied, can be due to the reduction of alcohol, such as ethanol, used in washing steps of the disclosed method. In embodiments, cumulative repeated binding steps can reduce the total alcohol consumption up to 95%, 90%, 85%, 80%, 75%, 70%, 60%, 50%, 40%, 30%, 20%, 10%, 5% per unit of nucleic acid compared to the method where single binding step is applied. In embodiments the alcohol can be ethanol.

In embodiments, these successive binding steps can be performed either by successively adding magnetic particles and nucleic acids at each cycle (hereafter referred as the first option) or by placing all magnetic particles in the tube in one step and successively adding nucleic acids at each cycle (hereafter referred as second option). When the second option is chosen the magnetic particles added in one step should be in a sufficient concentration to have the sufficient binding capacity for the total amount of nucleic acids to be added in the following successive cycles. It should be obvious to the skilled person the ratio of magnetic particle concentration to nucleic acid in the first cycle of successive binding steps is higher in the second option than the first option. In embodiments, the magnetic particle concentration added in the first cycle of successive binding repeat is 20 times higher in the second option than the first option, for example 15 times higher, 10 times higher, 9 times higher, 8 times higher, 7 times higher, 6 times higher, 5 times higher, 4 times higher, 3 times higher, 2 times higher in the second option than the first option for the purification of the same amount of nucleic acids.

In specific embodiments, the method comprises further steps after the step c and prior to step d, wherein said further steps are
f. adding at least one nucleic acid of interest in a binding buffer to the modified magnetic particles;
g. separating the modified magnetic particles from the combined solution by applying a magnetic field;
wherein said steps f and g are successively repeated one or more times before proceeding with step d and e, preferably repeated 10 times.

More specifically, in such embodiments, the method may comprise:
combining a sample comprising at least one nucleic acid of interest in a binding buffer having a pH ranging from 5 to 10 with silica-based magnetic particles to form a solution;
incubating the solution for a time period sufficient to reversibly bind the at least one nucleic acid to the magnetic particles to form modified magnetic particles;
separating the modified magnetic particles from the combined solution by applying a magnetic field;
h. adding at least one nucleic acid of interest in a binding buffer to the modified magnetic particles;
i. separating the modified magnetic particles from the combined solution by applying a magnetic field;
wherein the binding buffer comprises at least one salt, present in a concentration ranging from 0.1 M to 5 M, at least one first alcohol, present in a concentration between 10% to 50% v/v and/or PEG at a concentration of 10% to 40% v/V,

Wherein the step i and j are successively repeated up to 20 times, preferably up to 10 times,
washing the at least one modified magnetic particle with at least one wash buffer comprising at least one second alcohol at a concentration of between 50% to 100%; and
j. combining the modified magnetic particle with an elution buffer in order to allow the elution of said nucleic acid of interest from said magnetic particle,
wherein said elution buffer has a pH ranging from about 5 to 10 and wherein said elution buffer has a conductivity of from 0.001 and 40 mS/cm or wherein the total salt concentration is from 0 to 50 mM.

In embodiments of the methods as taught herein, a sample comprising at least one nucleic acid can be combined with a binding buffer.

In embodiments, said binding buffer can have a pH ranging from 5 to 10, such as from 5 to 9, from 5.5 to 8.5, from 6 to 8, or from 6.4 to 7.5 and all ranges and subranges therein between. In embodiments, said binding buffer comprise of at least one first alcohol and/or PEG, at least one salt and at least one optional chelating agent such as EDTA.

The concentration of said at least one first alcohol in the binding buffer can range from 10% to 50%, from 10% to 40%, from 10% to 30%, from 10% to 20, from 15% to 20% v/v, including all ranges and subranges therebetween.

According to various embodiments, the at least one alcohol can be chosen from isopropanol, ethanol, methanol, butanol, and combinations thereof. In some embodiments, the at least one alcohol can be ethanol which is more suitable for the purpose of production of medicament when compared to commonly used isopropanol taught in prior art.

The concentration of PEG in the binding buffer can range from 10% to 40%, from 20 to 40%, from 20% to 35%, from 20% to 30% or from 25% to 35%, including all ranges and subranges therebetween. In an embodiment, 30% PEG is used.

In an embodiment, said PEG used in the binding buffer is chosen from PEG 600, PEG 1000, PEG 2000, PEG 3000, PEG 4000, PEG 6000, PEG 8000, PEG 10.000, PEG 20.000. In an embodiment, used PEG is PEG 8000.

The at least one salt can be present in the binding buffer in a concentration ranging from 0.1M to 5 M, for example, from 0.1 to 4M, from 0.1M to 3M, from 0.1M to 2M, from 0.1M to 1M, from 0.5 to 1M, from 0.5 to 2M, from 1M to 2M and from 2M and 3M and from 3M to 5M, including all ranges and subranges therebetween.

In embodiments at least one salt can be present in the binding buffer in a concentration ranging from 0.1M to 0.5M, from 0.5 M to 1.5M, from 0.5M to 5M, from 1M to 5M, from 2M to 5M, from 3M to 5M, from 4M to 5M, from 3M to 4M, from 1M to 3M, from 1M to 4M, from 1M to 1.5M, from 1M to 1.6M, from 1M to 1.7M, from 1M to 1.8M, including all ranges and subranges therebetween.

According to various embodiments, the at least one salt can be sodium chloride (NaCl).

In embodiments, said binding buffer is devoid of toxic chaotropic agents such as guanidine salts (guanidinium thiocyanate or guanidine thiocyanate), iodide, perchlorate and trichloroacetate, preferably guanidine salts. Majority of the methods in prior art, including magnetic bead purifications use guanidium salts such as guanidinium thiocyanate or guanidinium-HCl as common substance because of the extremely chaotropic nature of these chemicals. In embodiments of the methods as taught herein, replacing highly toxic and expensive guanidine thiocyanate with common salt sodium chloride (NaCl) gives the invention advantages in multiple aspects such as reduced cost, safer working environment, meeting the regulation for safe drug production, and faster RNA purification due to the reduce number of rounds of wash steps to eliminate toxic guanidinium thiocyanate from the sample.

As used herein the term “magnetic particle” and variations thereof is intended to denote a particle with a magnetic, e.g., paramagnetic or superparamagnetic, core coated with at least one material having a surface to which nucleic acid can reversibly bind. Suitable magnetic particles can include, for example, carboxyl coated paramagnetic particles, silica-based paramagnetic particles, and the like. In embodiments described herein, the magnetic particles are silica-based magnetic particles wherein said silica-based particles can comprise, in some embodiments, a paramagnetic core coated with siliceous oxide, thus providing a hydrous siliceous oxide adsorptive surface to which nucleic acid can bind (e.g., a surface comprising silanol groups). In other or further embodiments, the magnetic particles can be coated with ligand which interacts with nucleic acids or can be surface-modified to produce functionalized surfaces, such as weakly or strongly positively charged, weakly or strongly negatively charged, or hydrophobic surfaces, to name a few.

The magnetic particles can be present in the binding buffer in a concentration ranging, for instance, from about 0.1 μg/μl to about 60 μg/μl, such as from about 0.5 μg/μl to about 55 μg/μl, from about 1 μg/μl to about 50 μg/μl, from 0.1 μg/μl to μg/μl, from 1 μg/μl to 15 μg/μl, 5 μg/μl to 10 μg/μl, from about 2 μg/μl to about μg/μl, from about 3 μg/μl to about 40 μg/μl, from about 4 μg/μl to about 35 μg/μl, from about 5 μg/μl to about 30 μg/μl, from about 6 μg/μl to about 25 μg/μl, from about 7 μg/μl to about 20 μg/μl, from about 8 μg/μl to about 15 μg/μl, or from about 9 μg/μl to about 10 μg/μl, including all ranges and subranges therebetween.

In specific embodiments, such as where the repeated binding steps are preferred, additional magnetic particles can be added to the binding buffer. It is clear that the further addition of magnetic beads will increase the final concentration of magnetic beads in binding buffer. In embodiments, the additional magnetic particles added to the binding buffer can result in the magnetic bead concentration in said binding buffer up to 20 times, for example 15 times, 10 times, 5 times, 2 times more than 60 μg/μl, as disclosed above. For example, the final one magnetic particle concentration present in the binding buffer can range from 0.1 μg/μl to 1200 μg/μl, for instance, from 1 μg/μl to 1100 μg/μl, from 10 μg/μl to 1000 μg/μl, from 20 μg/μl to 800 μg/μl, from 30 μg/μl to 700 μg/μl, from 40 μg/μl to 600 μg/μl, 40 μg/μl to 500 μg/μl, from 50 μg/μl to 400 μg/μl, from 60 μg/μl to 300 μg/μl, from 70 μg/μl to 200 μg/μl, from 80 μg/μl to 150 μg/μl, from 600 μg/μl to 1200 μg/μl, from 700 μg/μl to 1200 μg/μl, from 800 μg/μl to 1200 μg/μl, from 900 μg/μl to 1200 μg/μl, from 1000 μg/μl to 1200 μg/μl, or 0.1 μg/μl to 1200 μg/μl, from 0.1 μg/μl to 1100 μg/μl, 0.1 μg/μl to 1000 μg/μl, from 0.1 μg/μl to 900 μg/μl, from 0.1 μg/μl to 800 μg/μl, 0.1 μg/μl to 700 μg/μl, from 0.1 μg/μl to 600 μg/μl, from 0.1 μg/μl to 500 μg/μl, 0.1 μg/μl to 400 μg/μl, from 0.1 μg/μl to 300 μg/μl, 0.1 μg/μl to 200 μg/μl, 0.1 μg/μl to 100 μg/μl, 1 μg/μl to 100 μg/μl, 10 μg/μl to 100 μg/μl, or 50 μg/μl to 100 μg/μl including all ranges and subranges therebetween.

In embodiments of the methods as taught herein, a volumetric ratio between the sample and the binding buffer can range, for example, from 1:1 to 1:3, such as from 1:1 to 1:1.5, or from 1:1.5 to about 1:2.5, including all ranges and subranges therebetween. Incubation time period for the mixed solution comprising sample comprising at least one nucleic acid of interest, binding buffer and silica-based magnetic particles can range from 0.1 minute to 30 minutes, from 0.1 minutes to 25 minutes, from 0.1 minutes to 20 min, from 0.1 to 10 minutes, or from 0.1 to 5 minutes, from 0.1 to 2 minutes including all ranges and subranges therebetween.

In specific embodiments, such as where the repeated binding steps are preferred, a volumetric ratio between the further added sample in a repeating step and the binding buffer can range, for example, from 1:1 to 1:3, such as from 1:1 to 1:1.5, or from 1:1.5 to about 1:2.5, including all ranges and subranges therebetween. It is clear that when repeating binding steps are preferred, most of the magnetic beads and most of the sample added in the preceding rounds of binding will remain in the tube in which the further sample in binding buffer is added. Most of the sample added in the preceding rounds of binding steps is expected to be attached to the magnetic particles. It will be obvious to a skilled person that the ratio of overall sample (including sample added in all rounds of binding steps and the last further added sample) present in reaction tube to the binding buffer will be higher than indicated above. A volumetric ratio between the overall sample and the binding buffer can range, for example, from 1:3 to 20:1, such as from 1:3 to 1:2, from 1:2 to 1:1, from 1:3 to 20:1, from 1:3 to 19:1, from 1:3 to 18:1, from 1:3 to 17:1, from 1:3 to 16:1, from 1:3 to 15:1, from 1:3 to 14:1, from 1:3 to 13:1, from 1:3 to 12:1, from 1:3 to 11:1, from 1:3 to 10:1, from 1:3 to 9:1, from 1:3 to 8:1, from 1:3 to 7:1, from 1:3 to 6:1, from 1:3 to 4:1, from 1:3 to 3:1, from 1:3 to 2:1 including all ranges and subranges therebetween.

It is believed that the salt(s) and alcohol(s) introduced by the binding buffer can enhance the ability of nucleic acid, such as RNA, to reversibly (e.g., non-covalently) bind to the surface of the magnetic particle, such as a silica surface. The magnetic particles thus modified, e.g., comprising reversibly bound nucleic acid, can then be separated from the unbound contaminants such as salts, proteins, enzymes, oligonucleotides, DNA templates, and nucleotide triphosphates (NTPs). For instance, a magnet can be placed in proximity to the modified magnetic particles and used to draw the particles together, e.g., to form an aggregate or pellet. In certain embodiments, a container, such as a tube, containing a combined solution comprising the modified magnetic particles, can be placed on a magnetic stand, which can gather and somewhat immobilize the particles while the remaining solution is removed.

Upon binding the nucleic acid to the magnetic particles and after separation of the modified magnetic particles using a magnet, the particles can then be combined, rinsed, or washed with one or more wash buffers. A wash buffer can comprise, for example, at least one alcohol, optionally at least one buffer compound. The modified magnetic particles can be rinsed once or multiple times with the wash buffer, such as once, twice, three times, or more. Any additional washing can employ the same or different compositions, concentrations, and/or volumetric amounts.

In embodiments of the methods as taught herein, said wash buffer comprises at least one second alcohol in a concentration ranging, for example, from 50% to 100% by volume/volume (v/v), such as from 55% to 95%, from 60% to 85%, or from 60% to 80% by v/v, including all ranges and subranges therebetween. In embodiments, the at least one second alcohol can be chosen from isopropanol, methanol, ethanol, butanol, and combinations thereof. The at least one second alcohol in wash buffer can be same or different than the one first alcohol in binding buffer. In some embodiments the at least one second alcohol can be ethanol. In non-limiting embodiments, the wash buffer optionally can have at least one salt. The optional at least one salt, if present, in the binding buffer can be in a concentration ranging from 0.1M to 5 M, for example, from 0.1M to 4M, from 0.1M to 3M, from 0.1M to 2M, from 0.1M to 1M, and from 1M to 2M, including all ranges and subranges therebetween.

The optional at least one salt, if present, in the binding buffer can be in a concentration ranging from 0.5M to 5 M, for example, from 1M to 5M, from 2M to 5M, from 3M to 5M, from 4M to 5M, from 2M to 4M, from 3M to 4M, from 2M to 3M and from 1M to 4M, including all ranges and subranges therebetween.

According to various embodiments, the at least one salt in wash buffer can be sodium chloride (NaCl). In non-limiting embodiments, the wash buffer optionally can have at least one buffer and/or salt compound (e.g., Tris, citrate and the like) in concentrations ranging from 1 mM to about 10 mM. In embodiments, modified particles can be washed one or more time with at least one of said wash buffer. For example, said modified particles can be washed once, twice, or more with the wash buffer with intervals of separation of the modified magnetic particles by using a magnet in between the washes.

After the washing step, after addition and removal of the wash buffer, modified magnetic particles with nucleic acid reversibly bound to the surface may be provided, which can be free or substantially free of contaminants such as salts, proteins, enzymes, etc. According to various embodiments, the modified magnetic particles thus produced can then be combined with one or more elution buffers to release the bound nucleic acid and separate it from the magnetic particles.

According to various embodiments, the elution buffer is a low conductivity solution wherein the conductivity of the buffer ranges from 0.001 to 40 mS/cm, more preferably from 0.01 to 40 mS/cm, from 0.1 to 40 mS/cm, from 0.5 to 40 mS/cm, more preferably from 0.5 to 30 mS/cm, from 0.5 to 20 mS/cm, from 0.5 to 10 mS/cm, including all ranges and subranges therebetween.

In another or further embodiment, said elution buffer comprises a salt concentration of between 0.01 and 50 mM, more preferably between 0.1 to 40 mM, more preferably between 0.1 and 30 mM, more preferably between 0.1 and 20 mM.

Possible salts include sodium citrate, sodium chloride, sodium phosphate, potassium chloride, potassium phosphate and combinations thereof.

The pH of the elution buffer can range, for example, from 5 to about 10, such as from 5.5 to about 9, from 6 to 8, or from 6.4 to about 7.5, including all ranges and subranges therebetween.

For example, in some cases elution buffer can comprise water; in others water and EDTA, or only Tris, or Tris and EDTA, or Sodium citrate, or phosphate buffer, or Phosphate-buffered saline (PBS). The concentration of the sodium citrate, if used as elution buffer can range from 0.5 mM to 10 mM, for example from 0.6 mM to 5 mM, from 1 mM to 2 mM, including all ranges and subranges therebetween. The pH of the Sodium citrate, if used as elution buffer can range from pH 5.4 to 7.5, from pH 6 to pH 7, from pH 6 to 6 pH 6.5, including all ranges and subranges therebetween. According to non-limiting embodiments, the elution buffer can comprise water or 10 mM Tris-HCl, 1 mM EDTA, PH 7.4, or 10 mM Tris-HCl, pH 7.4, or 1 mM citrate Na, PH 6.4.

In embodiments, said elution buffer is devoid of toxic chaotropic agents such as guanidine salts (guanidinium thiocyanate or guanidine thiocyanate), iodide, perchlorate and trichloroacetate, preferably guanidine salts.

In embodiments, at least one modified particle incubated with the elution buffer for a time period ranging from 30 seconds to 30 minutes, from 1 minute to 20 minutes, from 1 minute to 10 minutes including all the ranges and subranges therebetween.

The magnetic particles (no longer attached to the nucleic acid) can subsequently be removed from the solution, e.g., separated using a magnet, yielding a purified nucleic acid in solution as the final product. The modified magnetic beads can be incubated with the elution buffer additional rounds (e.g., once, twice, or more) for optimal elution of the purified nucleic acid. For example, the methods disclosed herein can be used to provide a purified RNA product. The methods disclosed herein can, in certain embodiments, provide a relative RNA yield between 80% to 100% and give sufficient purity. In embodiments of the methods as taught herein, the purified RNA integrity is conserved while reducing the use of hazardous agent(s) and making the technology affordable by reducing the cost.

In embodiments of the methods as taught herein, the nucleic acid of interest can be a DNA molecule, RNA molecule, or DNA/RNA hybrid molecule. The at least one nucleic acid can include, for example, genomic DNA, chromosomal DNA (cDNA), plasmid DNA (pDNA), total RNA, messenger RNA (mRNA), ribosomal RNA (rRNA), transfer RNA (tRNA), long noncoding RNA (lncRNA), small RNA and/or RNA/DNA hybrids. In various embodiments, the sample can be a PCR product comprising DNA. In some embodiments, the sample can be a post-in vitro transcription (IVT) mixture comprising RNA, wherein said IVT RNA can be 5′ capped or uncapped RNA. In various embodiments, sample comprising the at least one nucleic acid can be extracted from a biological sample, wherein said biological sample can have bacterial, viral, plant or animal origin. According to additional embodiments, the at least one nucleic acid to be purified can be RNA, e.g., total RNA.

The current disclosure therefore also relates to methods of in vitro transcription of RNA, wherein the resulting RNA undergoes at least one purification step with silica magnetic beads according to any of the embodiments described herein.

In embodiments of the methods as taught herein, method can be semi- or fully automated.

In embodiments of the methods as thought herein, method can be used to produce kits for nucleic acid purification, wherein the kits comprising a binding buffer, a wash buffer, and an elution buffer. In a preferred embodiment, a kit for nucleic acid purification is disclosed, comprising:
a) a binding buffer having a pH ranging from 5 to 10; at least one salt being NaCl, present in a concentration ranging from 0.1 M to 5 M and ethanol, present in a concentration between 10% to 50% volume/volume ratio and/or PEG at a concentration of 10% to 40% v/v.

Herein said ratio of said ethanol is preferably between 10% to 50%, between 10% to 40%, between 10% to 30%, between 10% to 20, and more preferably between 15% to 20% v/v, including all ranges and subranges therebetween.

The concentration of PEG in the binding buffer is preferably between 10% to 40%, between 20 to 40%, between 20% to 35%, between 20% to 30% or between 25% to 35%, including all ranges and subranges therebetween.
b) a wash buffer comprising at least 60% by volume of ethanol and optionally at least one buffer and/or salt compound.
c) an elution buffer chosen from water, solutions comprising at least one buffer compound with a conductivity of between 0.001 and 40 mS/cm or wherein the total salt concentration is from 0 mM to 50 mM.

In an embodiment, the kit further comprises a solution of magnetic particles. In embodiments, said buffers in said kit, especially the binding and elution buffer are devoid of toxic chaotropic agents such as guanidine salts (guanidinium thiocyanate or guanidine thiocyanate), iodide, perchlorate and trichloroacetate, preferably guanidine salts.

It will be clear to a skilled person that the embodiments as described in the context of the methodology above also apply to the kit as described herein and are incorporated herein.

### Examples and Description of Figures

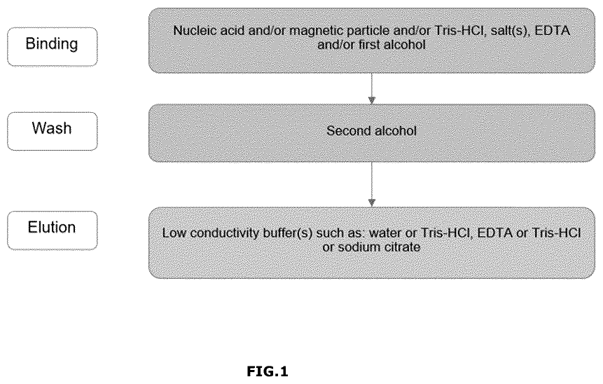

FIG. 1 shows a step sequence (binding, washing and elution) work-flow of nucleic acid purification method according to embodiments of the present invention.

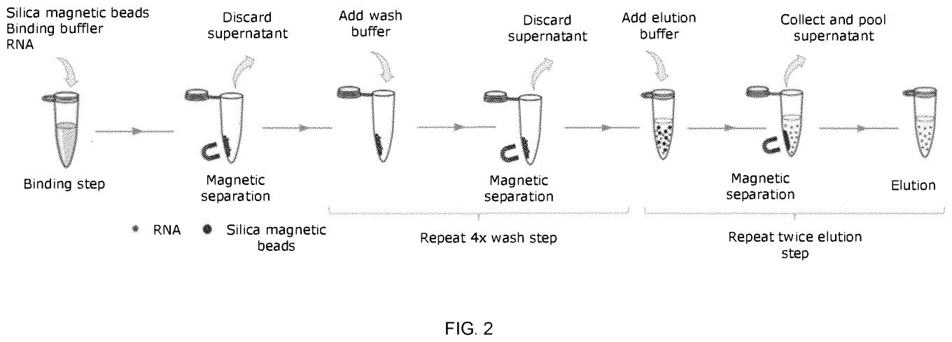

FIG. 2 shows a schematic process of nucleic acid purification method according to embodiments of the present invention. The process comprises:
a binding step where the coated silica magnetic particles, IVT sample with nucleic acids and binding buffer are mixed together. The obtained mixture is incubated to allow the reversible binding of the nucleic acids with the magnetic particles;
   a magnetic separation step where a magnetic field is applied to separate magnetic particles with nucleic acids;
   a washing step where a washing buffer comprising alcohol (such as Ethanol) is added and a magnetic field is applied once more to separate magnetic particles from the washing buffer which is discarded. The washing step can be applied once, twice or more.
   Finally, the process involves an elution step where an elution buffer is added to release nucleic acids from the magnetic particles and amagnetic field is applied to isolate the magnetic particles from the eluant comprising nucleic acids. The elution step can be applied once, twice or more.

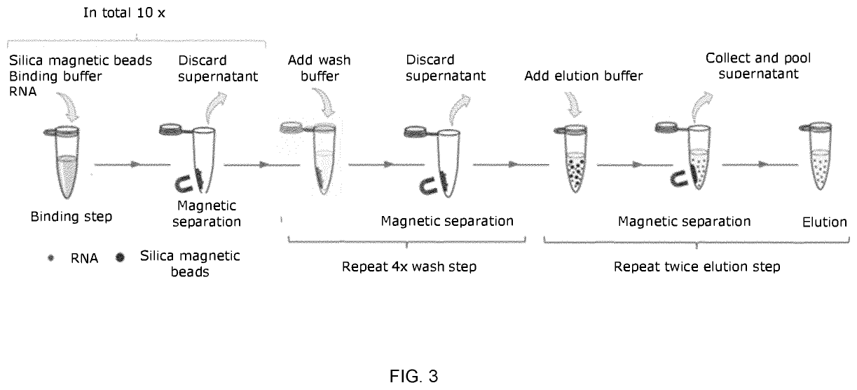

FIG. 3 shows a process of a nucleic acid purification method according to embodiments of the present invention. In this example, the binding and magnetic separation step are repeated up to 10 times before proceeding with the next step. The washing step and elution step can be applied once, twice or more times.

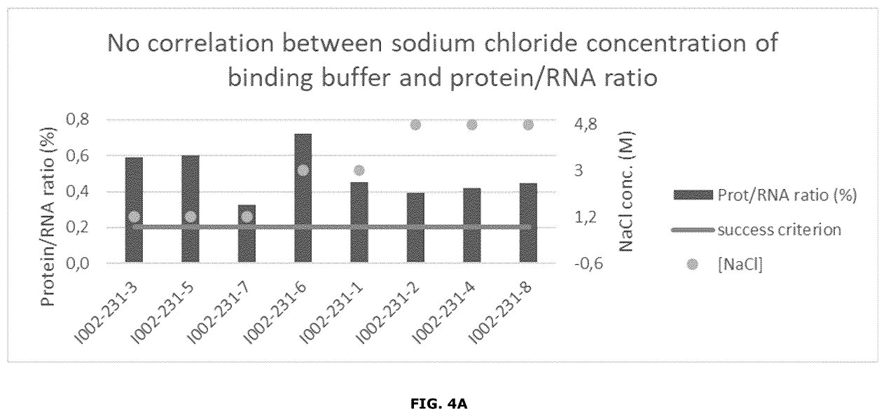

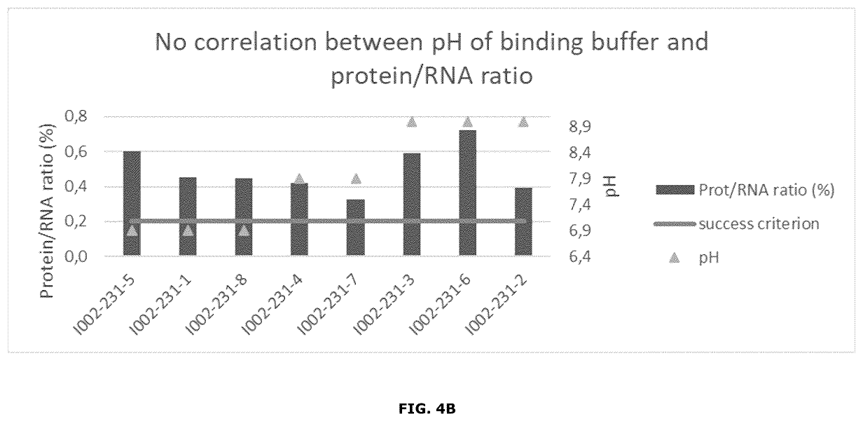

FIG. 4A and FIG. 4B presents a set of bar graphs, showing successful high quality RNA purification according to the method disclosed, using binding buffer in different NaCl concentrations and pH. 4A) The RNA/protein ratio is not impacted by the salt ranges of binding buffer. 4B) The RNA/protein ratio is not impacted by the pH ranges of binding buffer.

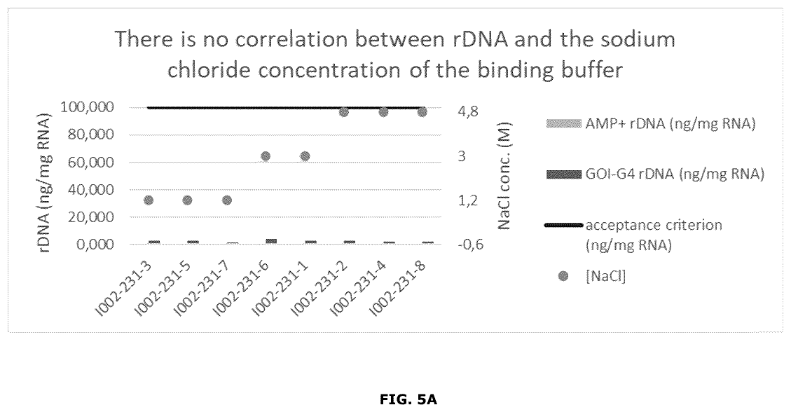

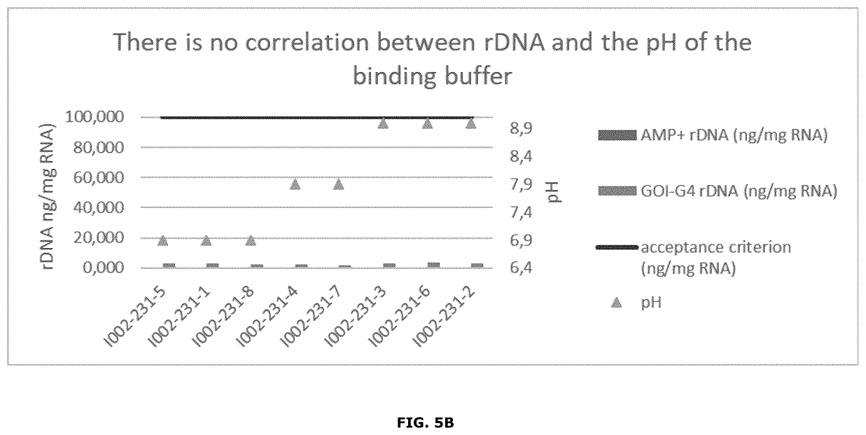

FIG. 5A and FIG. 5B presents a set of bar graphs, showing successful high quality RNA purification according to the method disclosed, using binding buffer in different NaCl concentrations and pH. 4A) rDNA content is not impacted by the salt ranges of binding buffer. 4B) rDNA content is not impacted by the pH ranges of binding buffer. rDNA (residual DNA), AMP+(Ampicillin Resistance gene), GOI-G4 (gene of interest).

### Example 1

A non-limiting example of possible buffers to use in conjunction with the methods as described herein include:

### Binding Buffer:

k. 13 mM Tris-HCl, 1.61 M NaCl, 1.3 mM EDTA, PH 7.4, 17% EtOH (or 13% EtOH)
l. 4M NaCl, 10 mM Tris-HCl, 1 mM EDTA, 30% PEG 8000, pH 8.0

### Washing Buffer:

m. Ethanol 60-80%
n. 10 mM Tris-HCl, 1 mM EDTA, 70% EtOH
   Elution Buffer: To be Chosen from the Solutions Listed Below
o. Water or
p. 10 mM Tris-HCl, 1 mM EDTA, PH 7.4 or
q. 5 mM Tris-HCl, PH 8.0 or
r. 10 mM Tris-HCl, PH 7.4 or
s. 1 mM citrate Na, PH 6.4

### Example 2

### Beads Preparation

A 3.5 ml RNA solution (2 mg RNA/ml) was mixed with an appropriate amount of silica magnetic beads, having a binding capacity of approximately 430 μg RNA/mg of beads.

In a pre-treatment step, beads were washed with RNAse free water. In short, RNAse free water was added to the beads, the solution was vortexed, and a magnetic field was applied for 120 seconds. The supernatant was subsequently removed.

In a subsequent step, beads were washed three times with a buffer solution comprising 40 mM Tris-HCl, 4.8 M NaCl, 4 mM EDTA, PH 7.4. The solution was vortexed and a magnetic field was applied for 120 seconds, after which the supernatant was removed.

### RNA Binding Step

The RNA solution resulting from an IVT was mixed with binding buffer without alcohol and with silica magnetic beads. After homogenizing of the solution, 100% EtOH was added, resulting in 17% of EtOH in final volume. Solution was mixed well to allow binding. Incubation time was at least 2 minutes. The binding took place at room temperature.

In a subsequent step, the beads were subjected to a magnetic field and the supernatant was removed. In a next step, the RNA-bound beads were washed two times with cold 80% EtOH.

In embodiments, the RNA-bound beads were washed with cold EtOH in concentrations between 50% and 100% EtOH, such as between 55% and 100% EtOH, 60% and 100% EtOH, 65% and 100% EtOH, 70% and 100% EtOH, 75% and 100% EtOH, 80% and 100% EtOH, 85% and 100% EtOH, 90% and 100% EtOH, 95% and 100% EtOH, and all the ranges and subranges there is in between.

In specific embodiments, the RNA-bound beads were washed more than two times, preferably 3 times, more preferably 4 times with ETOH.

Beads were homogenized by agitating for one minute at 1000 rpm. A magnetic field was applied for at least 120 seconds after which supernatant was removed.

Elution of the RNA occurred by adding an elution buffer having 1 mM Sodium citrate at pH 6.4. Incubation with the elution buffer was at least 5 minutes while the sample was agitated at 800 rpm at room temperature. Subsequently the supernatant was removed and the retrieved in an RNAse free tube. The elution protocol was repeated one additional time, and both elution samples were finally pooled.

### Example 3

DNA purification

50 μl of PCR product containing DNA was mixed with 35 μL of nuclease-free water and 100 μL of binding buffer containing 4M NaCl, 10 mM Tris-HCl, 1 mM EDTA, 30% PEG 8000, pH 8.0, bringing the total volume to 185 μl of DNA sample solution. 15 μL of magnetic beads were added the obtained DNA solution and mixed by vortex at least for 3 minutes. For the proper binding to take place, bead and DNA mix incubated on a tilt rotor at least for 2 minutes at room temperature.

In a subsequent step, the beads were subjected to a magnetic field for up to 3 minutes and the supernatant was removed. In a next step, the DNA-bound beads were washed three times with 700 μL of the wash buffer comprising 10 mM Tris-HCl, 1 mM EDTA, 70% EtOH. Beads were homogenized by vertexing for 3 minutes and incubated on a tilt rotor for 2 minutes. A magnetic field was applied for 3 minutes after which supernatant was removed. Tubes containing DNA-bound beads were kept on the magnetic stand for 10 minutes to air-dry the pellet of DNA-bound beads.

Elution of the DNA occurred by adding 10 μL elution buffer having5 mM Tris/HCl, PH 8.0. 3. The DNA-bound beads were resuspended in elution buffer by 5 minutes agitation at 800 rpm followed by 3 minutes incubation on the magnetic stand.

Subsequently the supernatant containing the DNA is collected and transferred to a new tube.

Similar results obtained when binding buffer comprise 10 to 40% v/v EtOH instead of PEG.

### Example 4

### Nucleic Acid Purification Process

In another example of nucleic acid purification, the binding and consequent magnetic separation steps are repeated up to 10 times (hereafter referred as successive binding steps) prior to the washing step. A schematic overview of such a process is given in FIG. 3.

As a comparative study, the method comprising successive binding steps (such a process is given in FIG. 3) was carried out along with the method with one round of binding (such a process given in FIG. 2). The yield (as amount purified nucleic acids) and the amount of buffers that were used in each method were compared.

### Buffers:

Buffer compositions as disclosed in Example 1 are used. The proportion of EtOH can vary from 13% to 100% in the Biding buffer and 50 to 100% in washing buffer.

### Method:

The coated silica magnetic beads, an IVT sample with nucleic acids and binding buffer were mixed together. The obtained mixture was incubated to allow the reversible binding of the nucleic acids to the beads. Subsequently, a magnetic field was applied to separate the magnetic particles with bound nucleic acids from the liquid phase, which was discarded or kept for other use. These successive binding steps were repeated up to 10 times. It was found that this repetition drastically reduced the use of washing and elution buffers and therefore drastically reduced the amount of organic solvent, such as EtOH, needed.

These successive binding steps were performed with two different approaches.

These were either:
(i) successively adding magnetic beads+nucleic acids at each cycle to the magnetic beads of a previous step, or
(ii) placing all magnetic beads in the tube from the start (first round) and successively adding nucleic acids at each cycle.

Both successive binding steps lead to similar results. The binding step was then followed by up to four successive washing steps with washing buffer. In some examples, the number of washing steps was refined to one or more than two. Samples were eluted after washing step. To increase the yield of collected nucleic acids the elution step was repeated.

### Results:

The results indicated that for the same number of nucleic acids, the process as described in this example comprising 10 cumulative binding reactions allowed to reduce the organic solvent consumption by 82% per unit of purified mRNA.

Table 1 present the use of ethanol in the purification of 38.4L of IVT with two embodiments of present invention.

TABLE 1

| | Baseline process | |
|---|---|---|
| Purification of | without successive | Process comprising |
| 38.4 L of IVT | binding steps | 10 binding cycles |
| Alcohol volume used | 15 L | 15 L |
| for the binding step | | |
| Alcohol volume used | 150 L | 15 L |
| for the wash step | | |
| Alcohol volume used | 165 L | 30 L |
| for the whole process | | |

The present invention is in no way limited to the embodiments described in the examples and/or shown in the figures. On the contrary, methods according to the present invention may be realized in many different ways without departing from the scope of the invention.

### Example 5: Quality of Purified Nucleic Acid Maintained in Various Salt Concentrations and Various pH of Binding Buffer

RNA resulting from an IVT reaction was purified using different binding buffer compositions and/or pH. Besides that, the method was carried out according to the method disclosed in example 2.

The experiments were carried out with 3 different NaCl concentrations, 0.4 (A), 1.0M (B), and 1.6 M (C) NaCl in the final binding reaction composition and in three different pH, 6.9, 7.9, and 9.0.

Quality and functional activity of the purified RNA were assessed by determining the Protein/RNA ratios, the target DNA content retrieved in the purified RNA bulk, and the capping efficiency of each purified RNA samples.

### RNA/Protein Ratio:

The purification quality is maintained in tested salt concentrations and/or pH of the binding buffer (see Table 2 below). Confirming the technical effect in the pH and salt ranges of a binding buffer according to disclosure.

For each purified RNA sample, the protein content (by using μBCA (micro BiCinchoninic acid Assay)) and RNA content (by using Nanodrop) were measured. The results showed no significant correlation between the salt concentration of the binding buffer and the protein/RNA ratio as depicted in FIG. 4A. FIG. 4B presents similar results that were obtained with variable pH conditions: no significant correlation between the tested pH of binding buffer and protein/RNA ratio is detected (also see Table 2 below).

TABLE 2

| Binding | | |
|---|---|---|
| buffer parameters | Prot/RNA | success |

| NaCl | pH | Batch | ratio (%) | criterion |
|---|---|---|---|---|
| A | 9.0 | I002-231-3 | 0.6 | 0.2 |
| A | 7.9 | I002-231-7 | 0.3 | 0.2 |
| A | 6.9 | I002-231-5 | 0.6 | 0.2 |
| B | 6.9 | I002-231-1 | 0.5 | 0.2 |
| B | 9.0 | I002-231-6 | 0.7 | 0.2 |
| C | 7.9 | I002-231-4 | 0.4 | 0.2 |
| C | 6.9 | I002-231-8 | 0.4 | 0.2 |
| C | 9.0 | I002-231-2 | 0.4 | 0.2 |

rDNA Content:

Another quality measurement for purified RNAs is the residual DNA (rDNA) content in the purified RNA bulk. rDNA content was measured in each purified RNA sample by using ELISA and the outcome is compared. The results showed that the method as disclosed here is equivalently effective in removing DNA from RNA sample thus in the purification of RNA in tested salt concentrations (FIG. 5A) and pH (FIG. 5B) of the binding buffer, as the tested variations did not have an impact on both targeted DNA genes retrieved in the purified RNA bulk.

### Capping Efficiency:

The capping efficiency of the RNAs purified by using different binding buffers according to disclosure was assessed by HPLC. Results showed that the capping efficiency of purified RNA samples was similar when different binding buffers were used (see Table 3 below).

| | |
|---|---|
| Binding | |
| buffer parameters | Capping |

| [NaCl] | pH | Batch | efficiency (%) |
|---|---|---|---|
| A | 9.0 | I002-231-3 | 88.6 |
| A | 6.9 | I002-231-5 | 88.0 |
| A | 7.9 | I002-231-7 | 87.3 |
| B | 6.9 | I002-231-1 | 89.5 |
| B | 9.0 | I002-231-6 | 87.6 |
| C | 9.0 | I002-231-2 | 89.1 |
| C | 7.9 | I002-231-4 | 87.9 |
| C | 6.9 | I002-231-8 | 88.7 |

Concluding, the results show that the method according to the present disclosure is successful in purifying nucleic acid when a binding buffer with a wide range of salt concentration and/or pH is used. The results clearly indicate that the tested range of sodium chloride concentrations and/or pH in the binding buffer is effective for purifying RNA with maintained quality requirements, such as RNA/protein ratio, rDNA content, and RNA capping efficiency.

1. A method for nucleic acid purification comprising:
a) combining a sample comprising at least one nucleic acid of interest in a binding buffer having a pH ranging from 5 to 10 with silica-based magnetic particles to form a solution;
wherein the binding buffer comprises at least one salt, present in a concentration ranging from 0.1 M to 5 M, at least one first alcohol present in a concentration between 10% to 50% v/v and/or polyethylene glycol (PEG) in a concentration range of 10% to 40% (v/v)
b) incubating the solution for a time period sufficient to reversibly bind the at least one nucleic acid to the magnetic particles to form modified magnetic particles;
c) separating the modified magnetic particles from the combined solution by applying a magnetic field;
d) washing the at least one modified magnetic particle with at least one wash buffer comprising at least one second alcohol at a concentration of between 60% to 100%, and
e) combining the modified magnetic particle with an elution buffer in order to allow the elution of said nucleic acid of interest from said magnetic particle, wherein said elution buffer has a pH ranging from about 5 to 10 and wherein said elution buffer has a conductivity of between 0.001 and 40 mS/cm or wherein the total salt concentration is from 0 to 50 mM.

2. The method according to claim 1, wherein steps a to c are repeated one or more times before proceeding to step d.

3. The method according to claim 2, wherein steps a to c are repeated 1 to 10 times.

4. The method according to claim 1, adding a further sample comprising the at least one nucleic acid of interest in the binding buffer and optionally non-modified silica-based magnetic particles to the modified magnetic particles of a preceding step c to form a solution, and repeating steps b and c.

5. The method according to claim 1, wherein the washing in step d is repeated one or more times, wherein the wash buffer is removed from the modified beads after each washing step.

6. The method according to claim 1, wherein the elution is successively repeated one or more times.

7. The method according to claim 1, wherein the concentration of said at least first alcohol is between 15% to 20% v/v.

8. The method according to claim 1, wherein said salt of said binding buffer is NaCl, wherein the concentration of NaCl is preferably between 0.5M and 2M.

9. The method according to claim 1, wherein said binding buffer is devoid of guanidinium salts.

10. The method according to claim 1, wherein said first alcohol in said binding buffer is chosen from isopropanol, methanol, ethanol, butanol, and/or combinations thereof.

11. The method of claim 1, wherein the wash buffer comprises 60% to 80% by volume of the at least one second alcohol, wherein said at least one second alcohol is chosen from isopropanol, methanol, ethanol, butanol, or combinations thereof.

12. The method according to claim 1, wherein the elution buffer is chosen from water or at least one pH buffer solutions, present in concentrations 8 mM to 12 mM and/or at least one ion chelating agent, present in concentrations from 0.8 mM to 1.2 mM.

13. The method of claim 1, where in the elution buffer is sodium citrate.

14. The method of claim 1, wherein said elution buffer is devoid of guanidinium salts.

15. The method of claim 1, wherein the nucleic acid of interest is DNA or RNA, more preferably mRNA resulting from an in vitro transcription (IVT) reaction.

16. The method of claim 1, wherein the at least one magnetic particle is coated with one or more ligand interacting with said nucleic acid.

17. The method according to claim 1, wherein said method is semi- or fully automated.

18. The method of claim 1, wherein the at least one magnetic particle present in the binding buffer is in a concentration ranging from 0.1 μg/μl to 200 μg/μl.

19. The method of claim 1, wherein the volumetric ratio of the sample containing the at least one nucleic acid to the binding buffer ranges from 1:1 to 1:3.

20. A kit for nucleic acid purification, comprising:
a binding buffer having a pH ranging from 5 to 10; at least one salt being NaCl, present in a concentration ranging from 0.1 M to 5 M; ethanol, present in a concentration between 10% to 50% volume/volume ratio and/or PEG at a concentration of between 10% and 40%;
a wash buffer comprising at least 60% by volume of ethanol;
an elution buffer chosen from water, solutions comprising at least one buffer compound with a conductivity of between 0.001 and 40 mS/cm or wherein the total salt concentration is from 0 to 50 mM; and
a solution comprising magnetic particles.
